(19) United States
(12) Patent Application Publication
(10) Pub. No.: US 2018/0010159 A1
(43) Pub. Date: Jan. 11, 2018

(54) METHOD OF PRODUCING SACCHARIDES AND METHOD OF PRODUCING ETHANOL

(71) Applicant: JGC CORPORATION, Chiyoda-ku, Tokyo (JP)

(72) Inventors: Daisuke TANEDA, Higashi Ibaraki-gun (JP); Shohei OKINO, Higashi Ibaraki-gun (JP); Makoto IKEO, Higashi Ibaraki-gun (JP)

(21) Appl. No.: 15/545,212

(22) PCT Filed: Mar. 4, 2015
(86) PCT No.: PCT/JP2015/056323

§ 371 Date: Jul. 20, 2017

(51) Int. Cl. C12P 19/02; C12P 19/14; C12P 7/14

(52) U.S. Cl. CPC C12P 19/02; C12Y 302/01004; C12P 7/14; C12P 19/14

(57) ABSTRACT

A method of producing saccharides, in which fluidity of the slurry including biomass can be retained; the used amount of the degrading enzyme can be reduced; and high concentration saccharides can be produced, is provided. In addition, a method of producing ethanol, in which high concentration ethanol can produced, is provided. The method of producing saccharides includes the steps of: adding a cellulose degrading enzyme to a slurry including a biomass; and degrading the cellulose included in the biomass by the degrading enzyme to produce saccharide including glucose as a major component. The concentration of the cellulose is retained at 75 g/L or less in a mixed solution including the slurry, the degrading enzyme, and an adsorption inhibitor inhibiting adsorption of the degrading enzyme to a lignin contained in the biomass.

### TECHNICAL FIELD

The present invention relates to a method of producing saccharides, in which saccharides are produced from biomass, and a method of producing ethanol, in which ethanol is produced from saccharides obtained from biomass.

### BACKGROUND ART

In the cases of producing a sugar solution, the major component of which is glucose, by enzymatically saccharifying a pre-treated biomass; or producing ethanol by simultaneously saccharifying and fermenting by adding a degrading enzyme and fermenting microorganism together to a pre-treated biomass, manufacturing techniques of a highly concentrated sugar solution (100 g/L or more) and a highly concentrated ethanol (50 g/L or more) are needed for commercialization.

Conventionally, it is hard to produce a highly concentrated sugar solution (100 g/L or more) and a highly concentrated ethanol (50 g/L or more) by using the cellulosic biomass as the raw material. In order to produce the highly concentrated sugar solution and ethanol, it is needed to increase the amount of the biomass filling the reaction tank. This means a high solid content (slurry concentration) in the reaction tank. Thus, fluidity of the slurry including the biomass is reduced; and the efficiencies of saccharification and fermentation are reduced.

Reasons for the reduced saccharification and/or fermentation include: the degrading enzyme not being provided efficiently to the entire biomass due to the high solid concentration with less free water; and the environment with less free water being not suitable for growth of the fermenting microorganism.

In addition, since ethanol fermentation is exothermic reaction, the heat of reaction has to be removed. However, in the case where the slurry concentration in the reaction tank is high, in addition to the thermal conductivity, it becomes hard to circulate the slurry by a pump or to stir the slurry in the reaction tank. Thus, the inside of the reaction tack cannot be cooled efficiently.

Conventionally, methods described below are proposed to solve the above-described problem. For example, in a method, the solid concentration of the biomass slurry is reduced by adding a solution including a sugar solution to the slurry including the biomass in advance to obtain fluidity of the biomass slurry (for example, refer Patent Literature 1 (PTL 1)). In another method, the biomass and the degrading enzyme are fed in the reaction tank divided into several times (for example, refer Non Patent Literature 2 (NPL 1)). In the method in which the biomass and the degrading enzyme are fed in the reaction tank divided into several times, the initial solid concentration in the reaction tank is reduced. Thus, fluidity of the slurry is retained. In addition, by adding the remaining biomass and the degrading enzyme after the content of the initially added solid is dissolved in a certain extent, acceptable fluidity of the slurry is obtained in the entire period from beginning to the end of the reaction.

### RELATED ART DOCUMENTS

### Patent Literature

PTL 1: Japanese Unexamined Patent Application, First Publication No. 2011-182741 (A)

### DISCLOSURE OF INVENTION

### Problems to be Solved by the Present Invention

However, in the conventional methods of producing a sugar solution or ethanol, a large amount of the degrading enzyme is needed in the enzymatic saccharification of the biomass. One of reasons is that, in the method in which the solution including the sugar solution is added in advance to the slurry including the biomass, the initial sugar concentration becomes so high that the degrading enzyme is inhibited by sugars for the activity of the enzyme to be reduced. Another reason is that, in the method in which the biomass and the degrading enzyme are fed in the reaction tank divided into several times, the initial concentration of the degrading enzyme becomes so low that the saccharification rate of the biomass becomes slow. The degrading enzyme is very expensive. It is essential to reduce the amount of use of the degrading enzyme in order to put the method, in which ethanol is produced by utilizing the technology of producing a sugar solution from the biomass; and/or the technology of simultaneously saccharifying and fermenting the biomass, into practical use.

The present invention is made under the circumstance described above. The purpose of the present invention is to provide a method of producing saccharides, in which fluidity of the slurry including biomass can be retained; the used amount of the degrading enzyme can be reduced; and high concentration saccharides can be produced. In addition, a method of producing ethanol, in which high concentration ethanol can produced, is provided.

### Means to Solving the Problems

An aspect of the present invention is a method of producing saccharides including the steps of:

adding a degrading enzyme of any one of a cellulose and a hemicellulose to a slurry including a biomass; and

degrading at least any one of a cellulose and a hemicellulose included in the biomass by the degrading enzyme to produce saccharide including glucose as a major component, wherein

a concentration of at least any one of the cellulose and the hemicellulose is retained at 75 g/L or less in a mixed solution including the slurry, the degrading enzyme, and an adsorption inhibitor inhibiting adsorption of the degrading enzyme to a lignin contained in the biomass.

In the above-described method of producing saccharides, an entire amount of the degrading enzyme may be added in beginning of the reaction free of adding the degrading enzyme during the reaction.

Other aspect of the present invention is a method of producing ethanol including the steps of:

adding a degrading enzyme of any one of a cellulose and a hemicellulose and a fermenting microorganism to a slurry including a biomass;

degrading at least any one of a cellulose and a hemicellulose included in the biomass by the degrading enzyme to produce saccharide including glucose as a major component; and

producing ethanol from the saccharides, wherein

a concentration of at least any one of the cellulose and the hemicellulose is retained at 75 g/L or less in a mixed solution including the slurry, the degrading enzyme, the fermenting microorganism and an adsorption inhibitor inhibiting adsorption of the degrading enzyme to a lignin contained in the biomass.

In the above-described method of producing ethanol, an entire amount of the degrading enzyme may be added in beginning of the reaction free of adding the degrading enzyme during the reaction.

### Effects of the Invention

According to the present invention, a method of producing saccharides, in which fluidity of the slurry including biomass can be retained; the used amount of the degrading enzyme can be reduced; and high concentration saccharides can be produced, is provided. In addition, a method of producing ethanol, in which high concentration ethanol can produced, is provided.

### BRIEF DESCRIPTION OF THE DRAWINGS

FIG. 1 is a graph showing the concentration of glucose contained in a mixed solution (aqueous solution) with time elapsed from the start of the reaction in the enzymatic saccharification step of the biomass.

FIG. 2 is a graph showing the concentration of glucose contained in a mixed solution (aqueous solution) with time elapsed from the start of the reaction in the enzymatic saccharification step of the biomass.

FIG. 3 is a graph showing the concentration of alcohol contained in a mixed solution (aqueous solution) with time elapsed from the start of the reaction in the simultaneous saccharification fermentation step of the biomass.

FIG. 4 is a graph showing the concentration of alcohol contained in a mixed solution (aqueous solution) with time elapsed from the start of the reaction in the simultaneous saccharification fermentation step of the biomass.

### EMBODIMENTS OF THE INVENTION

Embodiments of the method of producing saccharides and the method of producing ethanol, which are aspects of the present invention, are explained below.

The embodiments of the present invention are for only specifically explaining for better understanding of the scope of the present invention, and are not for limiting the present invention unless otherwise noted.

### [Method of Producing Saccharides]

The method of producing saccharides of the present invention includes the steps of adding a degrading enzyme of any one of a cellulose and a hemicellulose to a slurry including a biomass; and degrading at least any one of a cellulose and a hemicellulose included in the biomass by the degrading enzyme to produce saccharide including glucose as a major component, wherein a concentration of at least any one of the cellulose and the hemicellulose is retained at 75 g/L or less in a mixed solution including the slurry, the degrading enzyme, and an adsorption inhibitor inhibiting adsorption of the degrading enzyme to a lignin contained in the biomass.

In the method of producing saccharides of the present invention, biomass (at least any one of cellulose and hemicellulose) is added to the slurry divided into multiple times in order to retain fluidity of the slurry including the biomass. In other words, in the method of producing saccharides of the present invention, the enzymatic saccharification reaction of the biomass is performed in the state where the concentration of at least any one of the cellulose and the hemicellulose is retained at 75 g/L or less in a mixed solution including the slurry, the degrading enzyme, and an adsorption inhibitor from the beginning to the end of the enzymatic saccharification step, in which any one of cellulose and hemicellulose in the biomass is enzymatically saccharified by the degrading enzyme; and saccharides including glucose as the major component are obtained.

First, a pre-treatment is performed to the biomass (woods, weeds, or crop residues) in the method of producing saccharides of the present embodiment. Contact efficiency between: at least any one of cellulose and hemicellulose included in the biomass; and the degrading enzyme of at least any one of cellulose and hemicellulose, is improved by performing the pre-treatment. In the present embodiment, “at least any one of cellulose and hemicellulose included in the biomass” means “cellulose” or “a mixture of cellulose and hemicellulose.” In addition, “the degrading enzyme of at least any one of cellulose and hemicellulose” means “a cellulose degrading enzyme” or “an enzyme degrading cellulose and hemicellulose” in the present embodiment.

The pre-treatment includes the alkali treatment, the organic solvent treatment, the steam blasting treatment, the dilute sulfuric acid treatment and the like to the biomass. However, from the standpoint of the enzymatic saccharification yield and equipment costs, the steam blasting treatment, the alkali treatment or the dilute sulfuric acid treatment can be suitably utilized.

As the alkali treatment, the organic solvent treatment, the dilute sulfuric acid treatment, and the steam blasting treatment to the biomass, the known treatments can be utilized.

Next, the slurry including biomass (hereinafter, referred as “the biomass slurry” occasionally) is prepared by dispersing the pre-treated biomass in a solution (solvent).

The concentration of the biomass slurry, which is the concentration of the biomass in the biomass slurry, is appropriately adjusted depending on the kind of the biomass, the pre-treatment method or the like. However, it is preferable that the concentration is adjusted in such a way that the amount of at least one of cellulose and hemicellulose in the biomass slurry is 50 g/L to 75 g/L.

As the solution (solvent) used for preparing the biomass slurry, water can be named, for example.

As long as the concentration of the biomass in the biomass slurry is within the above-described range, a moderate amount of the free solution exists without being absorbed entirely in the fine pores of the biomass. Thus, the degrading enzyme can move freely in the biomass slurry. In addition, reactivity between the degrading enzyme and at least any one of cellulose and hemicellulose is improved, since stirring operation of the biomass becomes easy. In addition, if the amount of at least any one of cellulose and hemicellulose in the biomass slurry were less than 50 g/L, efficiency of forming saccharides including glucose as the major component would be deteriorated extensively. Thus, it is not preferable.

Next, the mixture A-1 including the biomass slurry, the degrading enzyme and the adsorption inhibitor is prepared by: feeding the tank with the biomass slurry, the solution (the enzyme solution) including the enzyme, which is suitable for degrading the cellulose included in the biomass slurry and an enzyme degrading an appropriate amount of any one of cellulose and hemicellulose, and the adsorption inhibitor which is for inhibiting adsorption of the degrading enzyme to the lignin included in the biomass; and mixing the biomass slurry, the enzyme solution and the adsorption inhibitor (the mixing step).

In this mixing step, the concentration of the biomass slurry in the mixed solution A-1 (the addition amount) is adjusted in such a way that the concentration of at least any one of cellulose and hemicellulose in the mixed solution A-1 becomes 75 g/L or less, preferably 35 g/L to 75 g/L, before the beginning of the reaction, which is before any one of cellulose and hemicellulose included in the biomass is degraded by the degrading enzyme.

In this mixing step, pH of the mixed solution A-1 including the biomass slurry, the degrading enzyme and the adsorption inhibitor is adjusted in such a way that pH becomes the most suitable pH condition for the degrading enzyme used. In addition, the temperature of the reaction tank is adjusted in such a way that the temperature becomes the most suitable temperature for the used degrading enzyme of at least any one of the cellulose and the hemicellulose.

In this mixing step, it is preferable that pH of the mixed solution A-1 is adjusted for the degrading enzyme to function actively. Specifically, it is preferable that pH is adjusted to 4 to 6.

In addition, it is preferable that the temperature of the mixed solution A-1 is adjusted for the degrading enzyme to function actively in the mixing step. Specifically, it is preferable that the temperature of the mixed solution A-1 is adjusted to 40° C. to 60° C.

As the degrading enzyme for degrading the biomass, a cellulase is used, for example. In the case where hemicellulose is included in the biomass, it is preferable that a xylanase or a mannanase is added in addition to the cellulase as the enzyme degrading the hemicellulose.

The addition amount of the degrading enzyme in the mixed solution A-1 (on a mass basis of the protein) is 0.4 part by mass to 2 parts by mass in the case where the mass of the biomass to be degraded is defined as 100 parts by mass. More preferably, the added amount of the degrading enzyme is 0.6 part by mass to 1.5 parts by mass.

As the adsorption inhibitor, the bovine serum albumin (BSA), the cheese whey, the grain-derived proteins and the like can be name, for example.

It is preferable that the addition amount of the adsorption inhibitor in the mixed solution A-1 is 0.1 part by mass to 2 parts by mass in the case where the amount of the biomass to be degraded is defined as 100 parts by mass. More preferably, the addition amount of the adsorption inhibitor is 0.4 part by mass to 1.5 parts by mass.

For stirring the mixed solution A-1 and the mixed solution B-1, which will be described later, stirring blades or the like are used.

By stirring and mixing the mixed solution A-1 (or the mixed solution B-1) gently enough not to excessively deactivate the degrading enzyme included in the mixed solution A-1 (or the mixed solution B-1) in the reaction tank, at least any one of cellulose and hemicellulose in the biomass is enzymatically saccharified efficiently by the degrading enzyme; and saccharides having glucose as the major component are obtained in the present embodiment (the enzymatically saccharifying step).

In this enzymatically saccharifying step, the biomass is added to the mixed solution A-1 sequentially, while the concentration of at least any one of cellulose and hemicellulose is retained at 75 g/L or less in the mixed solution A-1 including the biomass slurry, the degrading enzyme and the adsorption inhibitor in order to retain fluidity of the biomass slurry. In other words, the biomass is sequentially added to the mixed solution A-1 in such a way that the concentration of at least any one of cellulose and hemicellulose in the mixed solution A-1 is retained at 75 g/L or less by complementing the amount of at least any one of cellulose and hemicellulose reduced by being degraded and saccharified by the degrading enzyme.

It is preferable that: the entire amount of the degrading enzyme is added to the mixed solution A-1 in the beginning of the enzymatically saccharification reaction, practically in the beginning of the mixing step; and there is no (subsequent) addition of the degrading enzyme to the mixed solution A-1 in the enzymatically saccharification reaction. In this way, the concentration of the enzyme in the beginning of the reaction becomes high in the mixed solution A-1; and the biomass can be degraded efficiently.

In addition, in this enzymatically saccharifying step, it is preferable that the temperature of the mixed solution A-1 is adjusted for the enzyme to function actively. Specifically, it is preferable that the temperature of the mixed solution A-1 is retained to 40° C. to 60° C.

In this enzymatically saccharifying step, even in the case where the saccharides having glucose as the major component are produced, the concentration of the biomass slurry in the mixed solution B-1 (addition amount) is adjusted in such a way that the concentration of at least any one of cellulose and hemicellulose becomes 75 g/L or less in the mixed solution B-1 including the biomass slurry, the degrading enzyme, the absorption inhibitor and the saccharides having glucose as the major component.

The addition amount of the biomass slurry in the mixed solution B-1 is adjusted by measuring the concentration of the saccharides having glucose as the major component in the mixed solution B-1 (the concentration). The amount of saccharides, which have glucose as the major component and produced by at least any one of cellulose and hemicellulose being saccharified, (the produced amount) can be calculated based on the formula (1) indicated below.

Amount of glucose and xylose produced from at least any one of cellulose and hemicellulose (the produced amount) (g)=Decrease amount of at least any one of cellulose and hemicellulose (the amount converted to saccharides) (g)×1.1 (1)

In other words, by performing inverse calculation using the above-indicated formula (1), the decrease amount of at least any one of cellulose and hemicellulose (the amount converted to saccharides) can be calculated. Based on the obtained decrease amount and the volume of the mixed solution B-1, the addition amount of the biomass slurry to the mixed solution B-1 for the concentration of at least any one of cellulose and hemicellulose to be 75 g/L or less in the mixed solution B-1 can be determined.

According to the method of producing saccharides of the present embodiment, the concentration of at least any one of cellulose and hemicellulose is retained at 75 g/L or less in the mixed solutions A-1 and B-1 including the biomass slurry, the degrading enzyme and the adsorption inhibitor. Therefore, the saccharides having highly concentrated glucose (150 g/L or more) as the major component can be produced without using a large amount of the degrading enzyme, while fluidity of the biomass slurry-containing mixed solution A-1 or B-1 is retained at an acceptable extent.

### [Method of Producing Ethanol]

The method of producing ethanol of the present invention includes the steps of: adding a degrading enzyme of any one of a cellulose and a hemicellulose and fermenting microorganism to a slurry including a biomass; degrading at least any one of a cellulose and a hemicellulose included in the biomass by the degrading enzyme to produce saccharides, a major component of which is glucose; and producing ethanol from the saccharides, wherein a concentration of at least any one of the cellulose and the hemicellulose is retained at 75 g/L or less in a mixed solution including the slurry, the degrading enzyme, the fermenting microorganism and an adsorption inhibitor inhibiting adsorption of the degrading enzyme to a lignin contained in the biomass.

In the method of producing ethanol of the present invention, biomass (at least any one of cellulose and hemicellulose) is added to the slurry divided into multiple times in order to retain fluidity of the slurry including the biomass. In other words, in the method of producing saccharides of the present invention, the enzymatic saccharification reaction of the biomass is performed in the state where the concentration of at least any one of the cellulose and the hemicellulose is retained at 75 g/L or less in a mixed solution including the slurry, the degrading enzyme, the fermenting microorganism and an adsorption inhibitor from the beginning to the end of the enzymatic saccharification step, in which any one of cellulose and hemicellulose in the biomass is enzymatically saccharified by the degrading enzyme; and saccharides including glucose as the major component are obtained, while the obtained saccharides having glucose as the major component are converted to ethanol by the fermentation microorganism.

First, the pre-treatment is performed to the biomass in the method of producing ethanol of the present embodiment in the same way as the method of producing saccharides of the above-described embodiment.

Next, the slurry including biomass (hereinafter, referred as “the biomass slurry” occasionally) is prepared in the same way as the method of producing saccharides of the above-described embodiment.

Next, the mixture A-2 including the biomass slurry, the degrading enzyme, the fermenting microorganism and the adsorption inhibitor is prepared by: feeding the tank with the biomass slurry, the solution (the enzyme solution) including an appropriate amount of at least any one of a cellulose-degrading enzyme and a hemicellulose-degrading enzyme, each of which is suitable for degrading at least any one of cellulose and hemicellulose included in the biomass slurry, a solution including fermenting microorganism (the fermenting microorganism solution) and the adsorption inhibitor which is for inhibiting adsorption of the degrading enzyme to the lignin included in the biomass; and mixing the biomass slurry, the enzyme solution, the fermenting microorganism solution, and the adsorption inhibitor (the mixing step).

In this mixing step, the concentration of the biomass slurry in the mixed solution A-2 (the addition amount) is adjusted in such a way that the concentration of at least any one of cellulose and hemicellulose in the mixed solution A-2 becomes 75 g/L or less before the beginning of the reaction, which is before any one of cellulose and hemicellulose included in the biomass is degraded by the degrading enzyme.

In this mixing step, pH of the mixed solution A-2 including the biomass slurry, the degrading enzyme, the fermenting microorganism and the adsorption inhibitor is adjusted in such a way that pH becomes the most suitable pH condition for the degrading enzyme used. In addition, the temperature of the reaction tank is adjusted in such a way that the temperature becomes the most suitable temperature for the used degrading enzyme of at least any one of the cellulose and the hemicellulose.

In this mixing step, it is preferable that pH of the mixed solution A-2 is adjusted for the degrading enzyme and the fermenting microorganism to function actively. Specifically, it is preferable that pH is adjusted to 4 to 6.

In addition, it is preferable that the temperature of the mixed solution A-2 is adjusted for the degrading enzyme and the fermenting microorganism to function actively in the mixing step. Specifically, it is preferable that the temperature of the mixed solution A-2 is adjusted to 30° C. to 40° C.

As the degrading enzyme for degrading the biomass, the same enzyme as in the above-described method of producing saccharide of the embodiment is used.

The addition amount of the degrading enzyme in the mixed solution A-2 (on a mass basis of the protein) is 0.4 part by mass to 2 parts by mass in the case where the mass of the biomass to be degraded is defined as 100 parts by mass. More preferably, the added amount of the degrading enzyme is 0.6 part by mass to 1.5 parts by mass.

As the fermenting microorganism, yeast, Escherichia coli, Zymomonus, Corynebacterium, or the like is used.

It is preferable that the addition amount of the fermenting microorganism in the mixed solution A-2 is the turbidity of 1 to 10. The turbidity is the index representing the amount of the fermenting microorganism (the concentration). The higher the concentration, more turbid the mixed solution, indicating the amount of the fermenting microorganism by the degree of turbidity.

As the adsorption inhibitor, the same adsorption inhibitors as the method of producing saccharides of the above-described embodiment are used.

It is preferable that the addition amount of the adsorption inhibitor in the mixed solution A-2 is 0.1 part by mass to 2 parts by mass in the case where the amount of the biomass to be degraded is defined as 100 parts by mass. More preferably, the addition amount of the adsorption inhibitor is 0.4 part by mass to 1.5 parts by mass.

For stirring the mixed solution A-2; and the mixed solution B-2 and the mixed solution C-2, which will be described later, stirring blades or the like are used.

By stirring and mixing the mixed solution A-2 (the mixed solution B-2, or the mixed solution C-2) gently enough not to excessively deactivate the degrading enzyme and the fermenting microorganism included in the mixed solution A-2 (the mixed solution B-2, or the mixed solution C-2) in the reaction tank, at least any one of cellulose and hemicellulose in the biomass is enzymatically saccharified efficiently by the degrading enzyme; and saccharides having glucose as the major component are obtained, while the obtained saccharides having glucose as the major component are converted to ethanol by the fermenting microorganism in the present embodiment (the simultaneous saccharifying and fermenting step). The step in which the enzymatically saccharifying step and the fermenting step are performed at the same time is called as the simultaneous saccharifying and fermenting step.

In this simultaneous saccharifying and fermenting step, the biomass is added to the mixed solution A-2 sequentially, while the concentration of at least any one of cellulose and hemicellulose is retained at 75 g/L or less in the mixed solution A-2 including the biomass slurry, the degrading enzyme, the fermenting microorganism and the adsorption inhibitor in order to retain fluidity of the biomass slurry. In other words, the biomass is sequentially added to the mixed solution A-2 in such a way that the concentration of at least any one of cellulose and hemicellulose in the mixed solution A-2 is retained at 75 g/L or less by complementing the amount of at least any one of cellulose and hemicellulose reduced by being degraded and saccharified by the degrading enzyme.

It is preferable that: the entire amount of the degrading enzyme is added to the mixed solution A-2 in the beginning of the enzymatically saccharification reaction, practically in the beginning of the mixing step; and there is no (subsequent) addition of the degrading enzyme to the mixed solution A-2 in the enzymatically saccharification reaction. In this way, the concentration of the enzyme in the beginning of the reaction becomes high in the mixed solution A-2; and the biomass can be degraded efficiently.

In addition, in this simultaneous saccharifying and fermenting step, it is preferable that the temperature of the mixed solution A-2 is adjusted for the degrading enzyme and the fermenting microorganism to function actively. Specifically, it is preferable that the temperature of the mixed solution A-1 is retained to 30° C. to 40° C.

In this simultaneous saccharifying and fermenting step, even in the case where the saccharides having glucose as the major component are produced, the concentration of the biomass slurry in the mixed solution B-2 (addition amount) is adjusted in such a way that the concentration of at least any one of cellulose and hemicellulose becomes 75 g/L or less in the mixed solution B-2 including the biomass slurry, the degrading enzyme, the fermenting microorganism, the absorption inhibitor and the saccharides having glucose as the major component.

In addition, in this simultaneous saccharifying and fermenting step, even in the case where the saccharides having glucose as the major component and ethanol are produced, the concentration of the biomass slurry in the mixed solution C-2 (addition amount) is adjusted in such a way that the concentration of at least any one of cellulose and hemicellulose becomes 75 g/L or less in the mixed solution C-2 including the biomass slurry, the degrading enzyme, the fermenting microorganism, the absorption inhibitor, the saccharides having glucose as the major component and ethanol.

The addition amount of the biomass slurry in the mixed solution B-2 or the mixed solution C-2 is adjusted by measuring the concentration of ethanol in the mixed solution B-2 or the mixed solution C-2 (the concentration). The amount of ethanol produced by fermentation of the saccharides, which have glucose as the major component and produced by at least any one of cellulose and hemicellulose being saccharified, (the produced amount) can be calculated based on the formula (2) indicated below.

Amount of ethanol produced by fermentation of the saccharides which have glucose as the major component and produced by at least any one of cellulose and hemicellulose being saccharified (the produced amount) (g)=Decrease amount of at least any one of cellulose and hemicellulose (the amount converted to saccharides) (g)×1.1×0.51 (2)

The fermentation yield of ethanol is the parameter varying depending on the performance of the fermenting microorganism and is obtained by experimentation in advance.

In other words, by performing inverse calculation using the above-indicated formula (2), the decrease amount of at least any one of cellulose and hemicellulose (the amount converted to saccharides) can be calculated. Based on the obtained decrease amount and the volume of the mixed solution B-2 or the mixed solution C-2, the addition amount of the biomass slurry to the mixed solution B-2 or the mixed solution C-2 for the concentration of at least any one of cellulose and hemicellulose to be 75 g/L or less in the mixed solution B-2 or the mixed solution C-2 can be determined.

In addition, in this simultaneous saccharifying and fermenting step, it is preferable that the temperature of the mixed solution of A-2, B-2, or C-2 including the fermenting microorganism is adjusted for the fermenting microorganism to function actively. Specifically, it is preferable that the temperature is retained at 30° C. to 40° C.

This simultaneous saccharifying and fermenting step is performed until there is no progression of the reaction after the fermentation of the saccharides having glucose as the major component is progressed sufficiently by the fermenting microorganism. For example, the fermentation of the saccharides having glucose as the major component by the fermenting microorganism is performed for 1 day to 5 days at 30° C. to 40° C.

According to the method of producing ethanol of the present embodiment, the concentration of at least any one of cellulose and hemicellulose is retained at 75 g/L or less in the mixed solutions A-2, the mixed solution B-2, or the mixed solution C-2 including the biomass slurry, the degrading enzyme, the fermenting microorganism and the adsorption inhibitor. Therefore, highly concentrated ethanol (50 g/L or more) can be produced without using a large amount of the degrading enzyme, while fluidity of the biomass slurry-containing mixed solution A-2, B-1, or C-2 is retained at an acceptable extent.

### EXAMPLES

The present invention is explained in more detail by Examples below. However, the present invention is not limited by the descriptions of Examples below.

### Example 1

The steam blasted bagasse was mixed with an acetate buffer solution (50 mmol/L) having a pH of 5 to prepare each of the mixed solutions a-1 having the biomass slurry concentrations of 8 w/v % to 16 w/v % as shown in Table 1.

The mixed solutions b-1 including the biomass slurry, the degrading enzyme and the adsorption inhibitor were prepared by adding: cellulase in the amount of 4 mg per 1 g-dry of the biomass as the degrading enzyme; and the bovine serum albumin (BSA) in the amount of 10 mg per 1 g-dry of the biomass as the adsorption inhibitor, to each of the mixed solutions a-1 having different biomass slurry concentrations. By following the above-described procedures, the cellulose concentrations were adjusted in the range of 38.4 g/L to 76.8 g/L in the mixed solutions b-1 as shown in Table 1. By measuring the cellulose concentrations in the steam blasted bagasse, the cellulose concentrations in the mixed solutions b-1 were calculated.

Cellulose was degraded by the degrading enzyme by holding the mixed solutions b-1 at 50° C. for 20 days, while the mixed solutions b-1 were gently shaken by a shaker.

The concentrations of the saccharides, which were obtained by enzymatically degrading cellulose and have glucose as the major component, were measured by the high performance liquid chromatography (HPLC). In addition, the ratios (the saccharification ratios) of conversion from the cellulose to the saccharides having glucose as the major component were calculated based on: the amount of cellulose in the biomass; and the concentrations of glucose in the saccharified liquid. These results are shown in Table 1.

TABLE 1

| | Cellulose | | |
|---|---|---|---|
| | concentration | Concentration of | |
| Biomass slurry | in the | saccharides after the | Saccharification |
| concentration | mixed solution | reaction | ratio |
| (w/v %) | (g/L) | (g/L) | (%) |
| 16 | 76.8 | Not liquefied | Not liquefied |
| 15.6 | 75 | 58 | 70 |
| 15 | 71 | 66 | 85 |
| 14 | 67.2 | 63 | 86 |
| 12 | 57.6 | 54.5 | 86 |
| 10 | 48 | 46.5 | 88 |
| 8 | 38.4 | 38 | 90 |

Based on the results shown in Table 1, it is confirmed that the cellulose was degraded by the cellulase; and the saccharides having glucose as the major component were produced when the cellulose concentrations in the mixed solutions b-1 were 75 g/L or less. On the other hand, when the cellulose concentrations in the mixed solutions b-1 exceeded 75 g/L, the mixed solutions b-1 were not liquefied sufficiently, since the cellulose was not degraded by the cellulase; and the saccharides having glucose as the major component were not produced.

### Example 2

The steam blasted bagasse was mixed with an acetate buffer solution (50 mmol/L) having a pH of 5 to prepare the mixed solution a-2 having the biomass slurry concentration of 14 w/v %.

The mixed solutions b-2-1 including the biomass slurry, the degrading enzyme and the adsorption inhibitor were prepared by adding: cellulase in the amount of 4 mg per 1 g-dry of the biomass as the degrading enzyme; and the bovine serum albumin (BSA) in the amount of 10 mg per 1 g-dry of the biomass as the adsorption inhibitor, to the mixed solutions a-2. By following the above-described procedures, the cellulose concentration was adjusted to 67.2 g/L in the mixed solution b-2-1.

In addition, the mixed solution b-2-2 including the biomass slurry and the degrading enzyme was prepared by adding cellulase in the amount of 4 mg per 1 g-dry of the biomass as the degrading enzyme to the mixed solution a-2. By following the above-described procedures, the cellulose concentration was adjusted to 67.2 g/L in the mixed solution b-2-2.

By measuring the cellulose concentrations in the steam blasted bagasse, the cellulose concentrations in the mixed solution b-2-1 and the mixed solution b-2-2 were calculated.

Cellulose was degraded by the degrading enzyme by holding each of the mixed solutions b-2-1 and b-2-2 at 50° C. for 20 days, while the mixed solutions b-2-1 and b-2-2 were gently shaken by a shaker. In addition, after 5 days from the beginning of the reaction, the steam blasted bagasse was added in such a way that the cellulose concentrations of the mixed solution b-2-1 and the mixed solution b-2-2 became 70 g/L.

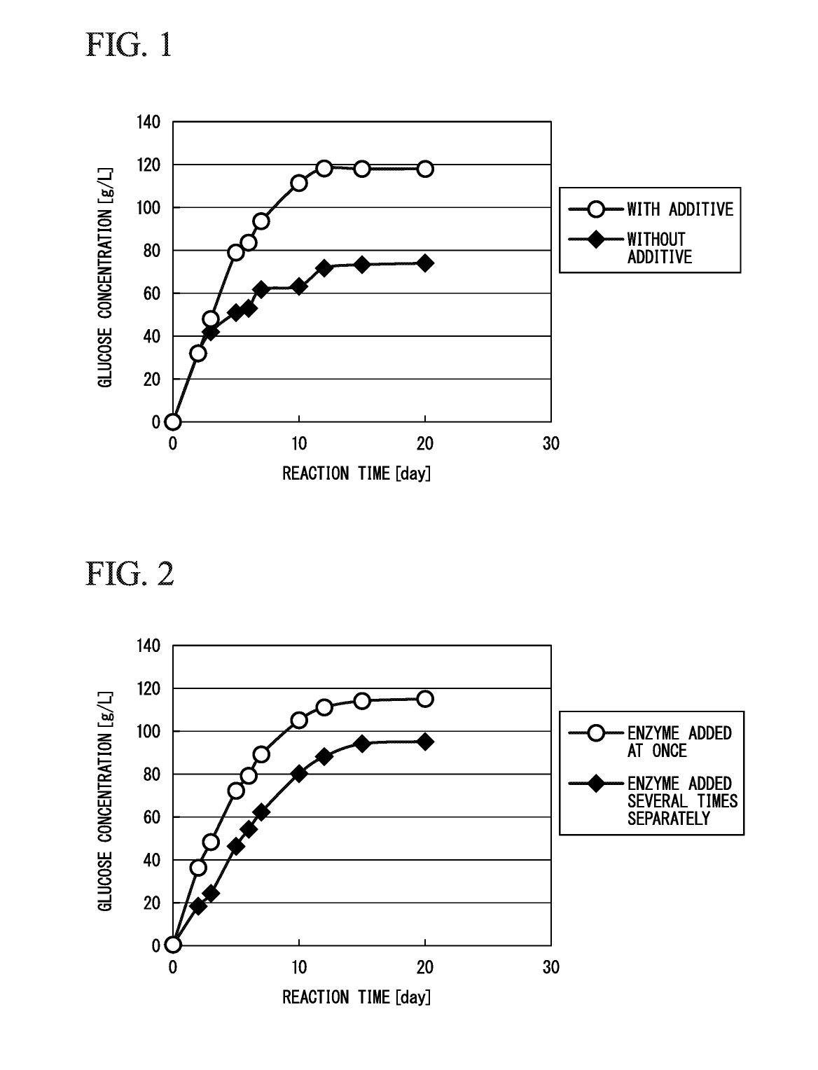

During the enzymatically degrading reaction, at predetermined time intervals, the concentrations of the saccharides, which were obtained by enzymatically degrading cellulose and have glucose as the major component, were measured by the high performance liquid chromatography (HPLC). Results are shown in FIG. 1.

Based on the results shown in FIG. 1, it was confirmed that the concentration of the saccharides having glucose as the major component reached to 118 g/L in the mixed solution b-2-1 including the absorption inhibitor, while the concentration of the saccharides having glucose as the major component was confined to 74 g/L in the mixed solution b-2-2 that was free of the adsorption inhibitor.

### Example 3

Five gram-dry of the steam blasted bagasse was mixed with an acetate buffer solution (50 mmol/L) having a pH of 5 to prepare the mixed solution a-3.

The mixed solutions b-3-1 including the biomass slurry, the degrading enzyme and the adsorption inhibitor were prepared by adding: cellulase in the amount of 40 mg per 1 g-dry of the biomass as the degrading enzyme; and the bovine serum albumin (BSA) in the amount of 10 mg per 1 g-dry of the biomass as the adsorption inhibitor, to the mixed solutions a-3. By following the above-described procedures, the cellulose concentration was adjusted to 60 g/L in the mixed solution b-3-1.

In addition, the mixed solution b-3-2 including the biomass slurry, the degrading enzyme and adsorption inhibitor was prepared by adding: cellulase in the amount of 20 mg per 1 g-dry of the biomass as the degrading enzyme; the bovine serum albumin (BSA) in the amount of 10 mg per 1 g-dry of the biomass as the adsorption inhibitor, to the mixed solution a-3. By following the above-described procedures, the cellulose concentration was adjusted to 60 g/L in the mixed solution b-3-2.

By measuring the cellulose concentrations in the steam blasted bagasse, the cellulose concentrations in the mixed solution b-3-1 and the mixed solution b-3-2 were calculated.

Cellulose was degraded by the degrading enzyme by holding each of the mixed solutions b-3-1 and b-3-2 at 50° C. for 20 days, while the mixed solutions b-3-1 and b-3-2 were gently shaken by a shaker. In addition, after 3 days from the beginning of the reaction, 5 g-dry of the steam blasted bagasse was added to the mixed solution b-3-1 and the mixed solution b-3-2. When the steam blasted bagasse was added, cellulase was added in the amount of 20 mg per 1 g-dry of the biomass as the degrading enzyme to the mixed solution b-3-2.

During the enzymatically degrading reaction, at predetermined time intervals, the concentrations of the saccharides, which were obtained by enzymatically degrading cellulose and have glucose as the major component, were measured by the high performance liquid chromatography (HPLC). Results are shown in FIG. 2.

Based on the results shown in FIG. 2, it was confirmed that the concentration of the saccharides having glucose as the major component reached to 115 g/L in the mixed solution b-3-1 in which 40 mg of the cellulase was added in the beginning of the reaction and was free of addition of cellulose during the reaction, while the concentration of the saccharides having glucose as the major component was confined to 95 g/L in the mixed solution b-3-2 in which 20 mg of the cellulase was added in the beginning of the reaction and 20 mg of the cellulase was further added during the reaction.

### Example 4

The steam blasted bagasse was mixed with an acetate buffer solution (50 mmol/L) having a pH of 5 to prepare each of the mixed solution a-4 having the biomass slurry concentrations of 8 w/v % to 16 w/v % as shown in Table 2.

The mixed solutions b-4 including the biomass slurry, the degrading enzyme, the adsorption inhibitor and the fermenting microorganism were prepared by adding: cellulase in the amount of 4 mg per 1 g-dry of the biomass as the degrading enzyme; the bovine serum albumin (BSA) in the amount of 10 mg per 1 g-dry of the biomass as the adsorption inhibitor; and yeast in the amount to obtain turbidity of 3 as the fermenting microorganism, to each of the mixed solutions a-4 having different biomass slurry concentrations. By following the above-described procedures, the cellulose concentrations were adjusted in the range of 38.4 g/L to 76.8 g/L in the mixed solutions b-4 as shown in Table 2. By measuring the cellulose concentrations in the steam blasted bagasse, the cellulose concentrations in the mixed solutions b-4 were calculated.

Cellulose was degraded by the degrading enzyme by holding the mixed solutions b-4 at 30° C. for 7 days, while the mixed solutions b-4 were gently shaken by a shaker. By following the procedure described above, the saccharides having glucose as the major component were produced; and ethanol was produced from the saccharides having glucose as the major component.

The concentrations of the ethanol in the mixed solutions b-4 were calculated by using the high performance liquid chromatography (HPLC). These results are shown in Table 2.

TABLE 2

| Biomass slurry | Cellulose concentration in | Ethanol concentration after |
|---|---|---|
| concentration | the mixed solution | the reaction |
| (w/v %) | (g/L) | (g/L) |
| 16 | 76.8 | Not liquefied |
| 15.6 | 75 | 20 |
| 15 | 71 | 29 |
| 14 | 67.2 | 28 |
| 12 | 57.6 | 24 |
| 10 | 48 | 20 |
| 8 | 38.4 | 17 |

Based on the results shown in Table 2, it is confirmed that the cellulose was degraded by the cellulase; the saccharides having glucose as the major component were produced; and the saccharides having glucose as the major component were degraded by the yeast to produce ethanol, when the cellulose concentrations in the mixed solutions b-4 were 75 g/L or less. On the other hand, when the cellulose concentrations in the mixed solutions b-4 exceeded 75 g/L, the mixed solutions b-4 were not liquefied sufficiently, since the cellulose was not degraded by the cellulase; and the saccharides having glucose as the major component were not produced. As a result, ethanol was not produced.

### Example 5

The steam blasted bagasse was mixed with an acetate buffer solution (50 mmol/L) having a pH of 5 to prepare the mixed solution a-5 having the biomass slurry concentration of 14 w/v %.

The mixed solutions b-5-1 including the biomass slurry, the degrading enzyme the adsorption inhibitor and the fermenting microorganism were prepared by adding: cellulase in the amount of 4 mg per 1 g-dry of the biomass as the degrading enzyme; the bovine serum albumin (BSA) in the amount of 10 mg per 1 g-dry of the biomass as the adsorption inhibitor; and yeast in the amount to obtain turbidity of 3 as the fermenting microorganism, to the mixed solutions a-5. By following the above-described procedures, the cellulose concentration was adjusted to 67.2 g/L in the mixed solution b-5-1.

In addition, the mixed solution b-5-2 including the biomass slurry, the degrading enzyme and the fermenting microorganism was prepared by adding cellulase in the amount of 4 mg per 1 g-dry of the biomass as the degrading enzyme; and yeast in the amount to obtain turbidity of 3 as the fermenting microorganism to the mixed solution a-5. By following the above-described procedures, the cellulose concentration was adjusted to 67.2 g/L in the mixed solution b-5-2.

By measuring the cellulose concentrations in the steam blasted bagasse, the cellulose concentrations in the mixed solution b-5-1 and the mixed solution b-5-2 were calculated.

Cellulose was degraded by the degrading enzyme by holding each of the mixed solutions b-5-1 and b-5-2 at 50° C. for 20 days to produce the saccharides having glucose as the major component, while the mixed solutions b-5-1 and b-5-2 were gently shaken by a shaker. From the saccharides having glucose as the major component, ethanol was produced. In addition, after 5 days from the beginning of the reaction, the steam blasted bagasse was added in such a way that the cellulose concentrations of the mixed solution b-5-1 and the mixed solution b-5-2 became 70 g/L.

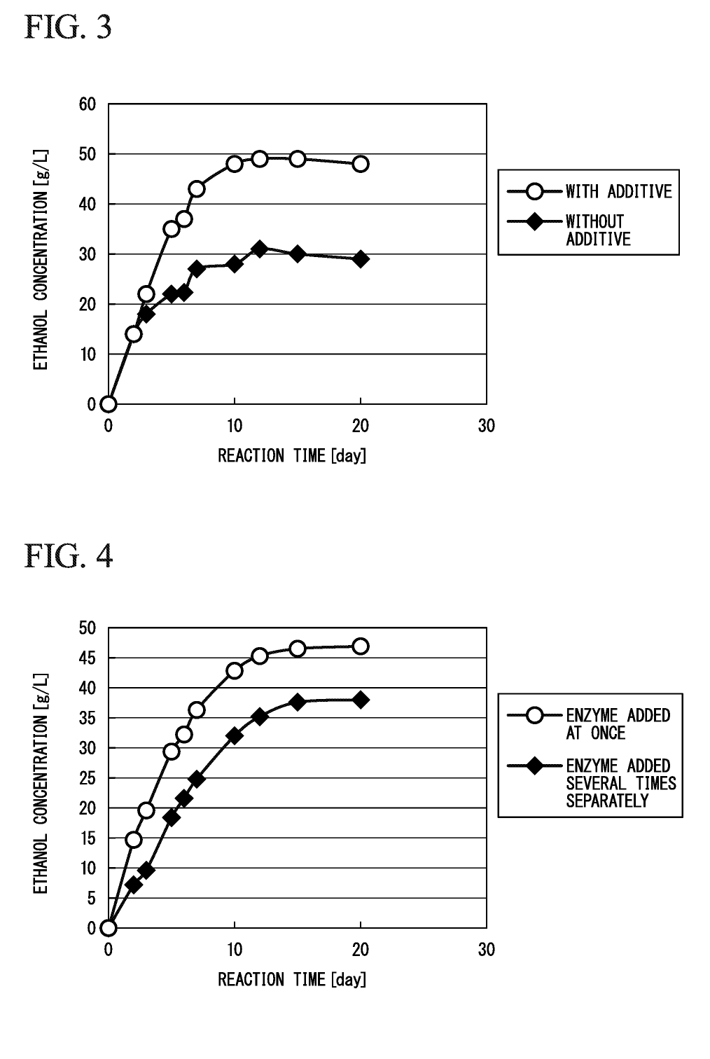

During the simultaneous saccharifying and fermenting reaction, at predetermined time intervals, ethanol concentrations were measured by the high performance liquid chromatography (HPLC). Results are shown in FIG. 3.

Based on the results shown in FIG. 3, it was confirmed that the ethanol concentration reached to 48 g/L in the mixed solution b-5-1 including the absorption inhibitor, while the ethanol concentration was confined to 29 g/L in the mixed solution b-5-2 that was free of the adsorption inhibitor.

### Example 6

Five gram-dry of the steam blasted bagasse was mixed with an acetate buffer solution (50 mmol/L) having a pH of 5 to prepare the mixed solution a-6.

The mixed solutions b-6-1 including the biomass slurry, the degrading enzyme, the adsorption inhibitor and the fermenting microorganism were prepared by adding: cellulase in the amount of 40 mg per 1 g-dry of the biomass as the degrading enzyme; the bovine serum albumin (BSA) in the amount of 10 mg per 1 g-dry of the biomass as the adsorption inhibitor; and yeast in the amount to obtain turbidity od 3 as the fermenting microorganism, to the mixed solutions a-6. By following the above-described procedures, the cellulose concentration was adjusted to 60 g/L in the mixed solution b-6-1.

In addition, the mixed solution b-6-2 including the biomass slurry, the degrading enzyme, adsorption inhibitor and the fermenting microorganism was prepared by adding: cellulase in the amount of 20 mg per 1 g-dry of the biomass as the degrading enzyme; the bovine serum albumin (BSA) in the amount of 10 mg per 1 g-dry of the biomass as the adsorption inhibitor; and yeast in the amount to obtain turbidity of 3 as the fermenting microorganism, to the mixed solution a-6. By following the above-described procedures, the cellulose concentration was adjusted to 60 g/L in the mixed solution b-6-2.

By measuring the cellulose concentrations in the steam blasted bagasse, the cellulose concentrations in the mixed solution b-6-1 and the mixed solution b-6-2 were calculated.

Cellulose was degraded by the degrading enzyme by holding each of the mixed solutions b-6-1 and b-6-2 at 50° C. for 20 days, while the mixed solutions b-6-1 and b-6-2 were gently shaken by a shaker. In addition, after 3 days from the beginning of the reaction, 5 g-dry of the steam blasted bagasse was added to the mixed solution b-6-1 and the mixed solution b-6-2. When the steam blasted bagasse was added, cellulase was added in the amount of 20 mg per 1 g-dry of the biomass as the degrading enzyme to the mixed solution b-6-2.

During the enzymatically degrading reaction, at predetermined time intervals, the concentrations of the saccharides, which were obtained by enzymatically degrading cellulose and have glucose as the major component, were measured by the high performance liquid chromatography (HPLC). Results are shown in FIG. 4.

Based on the results shown in FIG. 4, it was confirmed that the concentration of the ethanol concentration reached to 46 g/L in the mixed solution b-6-1 in which 40 mg of the cellulase was added in the beginning of the reaction and was free of addition of cellulose during the reaction, while the ethanol concentration was confined to 37 g/L in the mixed solution b-6-2 in which 20 mg of the cellulase was added in the beginning of the reaction and 20 mg of the cellulase was further added during the reaction.

### INDUSTRIAL APPLICABILITY

The present invention relates to a method of producing saccharides, in which fluidity of the slurry including biomass can be retained; the used amount of the degrading enzyme can be reduced; and high concentration saccharides can be produced. In addition, it relates to a method of producing ethanol, in which high concentration ethanol can produced.

1. A method of producing saccharides comprising the steps of:
adding a degrading enzyme of any one of a cellulose and a hemicellulose to a slurry including a biomass; and
degrading at least any one of a cellulose and a hemicellulose included in the biomass by the degrading enzyme to produce saccharide including glucose as a major component, wherein
a concentration of at least any one of the cellulose and the hemicellulose is retained at 75 g/L or less in a mixed solution including the slurry, the degrading enzyme, and an adsorption inhibitor inhibiting adsorption of the degrading enzyme to a lignin contained in the biomass.

2. The method of producing saccharides according to claim 1, wherein an entire amount of the degrading enzyme is added in beginning of the reaction free of adding the degrading enzyme during the reaction.

3. A method of producing ethanol comprising the steps of:
adding a degrading enzyme of any one of a cellulose and a hemicellulose and a fermenting microorganism to a slurry including a biomass;
degrading at least any one of a cellulose and a hemicellulose included in the biomass by the degrading enzyme to produce saccharide including glucose as a major component; and
producing ethanol from the saccharides, wherein
a concentration of at least any one of the cellulose and the hemicellulose is retained at 75 g/L or less in a mixed solution including the slurry, the degrading enzyme, the fermenting microorganism and an adsorption inhibitor inhibiting adsorption of the degrading enzyme to a lignin contained in the biomass.

4. The method of producing ethanol according to claim 3, wherein an entire amount of the degrading enzyme is added in beginning of the reaction free of adding the degrading enzyme during the reaction.
